(19) United States
(12) Patent Application Publication
(10) Pub. No.: US 2013/0071590 A1
(43) Pub. Date: Mar. 21, 2013

(54) MOLECULAR FILMS FOR HYDROPHOBIC IMPLANT SURFACES

(71) Applicant: Arizona Board of Regents, a body corporate acting, Scottsdale, AZ (US)

(72) Inventors: Nicole Herbots, Tempe, AZ (US); James Doug Bradley, Gilbert, AZ (US); Murdock Hart, Tempe, AZ (US); David Sell, Tempe, AZ (US); Shawn D. Whaley, Tempe, AZ (US); Qian Xing Bradley, Mesa, AZ (US)

(73) Assignee: Arizona Board of Regents, a body corporate acting for and on behalf of Arizona State University, Scottsdale, AZ (US)

(21) Appl. No.: 13/674,307

(22) Filed: Nov. 12, 2012

Related U.S. Application Data
(63) Continuation of application No. 13/503,485.
(60) Provisional application No. 61/259,298, filed on Nov. 9, 2009.

(51) Int. Cl. H01B 1/12; A61F 2/16; B65D 25/08

(52) U.S. Cl. CPC H01B 1/122; B65D 25/08; A61F 2/16
USPC 428/35.7; 252/500; 508/219; 428/532; 428/447; 428/452; 428/412; 428/36.9; 428/507; 206/219; 623/6.62; 427/2.24

(57) ABSTRACT

Compositions are disclosed containing a solvated viscoelastic polymeric gel diluted into an ionically conductive aqueous solution which can be usefully applied to any surface that is hydrophobic to act, for example, as an antifogging coating with minimal optical distortion and excellent transparency. The compositions can also be used as lubricious agents on medical implants, shunts, and surgical supplies to minimize tissue trauma, to maximize bio-compatibility, and to increase healing by enhancing better irrigation and flow in adjacent tissue.

### CROSS-REFERENCE TO RELATED APPLICATIONS

This application claim the benefit of the filing date of U.S. Provisional Application Ser. No. 61/259,298, filed Nov. 9, 2009, which is hereby incorporated by reference in its entirety.

### BACKGROUND OF THE INVENTION

Water condensation on silicone lenses during surgery has been identified and reported as a problem since 1994, as reported in Eaton et al. Ophthalmology. 1995; 102:733-736, and Carlson et al. Surv Ophthalmol. 1998; 42:417-437. For example, water can condense on the posterior surface of silicone intraocular lenses during fluid-air exchange. Such intraocular lens complications (IOL) can require removal or exchange.

The two existing approaches are:

(1) to use older materials, such as acrylic and PMMA. They do not exhibit condensation, but 25% of them become opaque over time, cancelling the benefit of cataract surgery; and

(2) to coat the silicone lens with a variety of viscoelastic gels. Such gels can distort the vision field when they are applied in thick coats making them highly impractical. Cataract Surgery Today noted in 2004, “[t]he use of viscoelastic and manual cleaning of condensation off the IOL are required to improve visualization, but the effect is suboptimal and transient.”

In addition, more recent acrylic lenses have been developed to overcome the limitations of acrylic surfaces when compared to silicon lenses, namely to eliminate growth of secondary cataracts on acrylic lenses (which does not occur with silicone lenses). These new acrylic lenses are called “hydrophobic”, and unfortunately, also exhibit similar condensation problems during post implantation surgery.

### SUMMARY OF THE INVENTION

In a first aspect, the present invention provides compositions, comprising a mixture of (a) a solvated viscoelastic polymeric gel, comprising a viscoelastic polymer having a molecular weight of between about 20,000 Da and about 4,000,000 Da; and (b) an ionically conductive aqueous solution; wherein the volume ratio of (a) to (b) is between 1:1 and 1:10.

In a second aspect, the present invention provides a device, comprising or consisting of (a) an optional first layer comprising or consisting of an ionically conductive aqueous solution; and (b) a second layer comprising or consisting of the composition of any embodiment of the first aspect of the invention in emulsion form; wherein when the first layer is present, the first layer and the second layer are in direct contact.

In a third aspect, the present invention provides kits comprising (a) a first container comprising or consisting of the viscoelastic component of any embodiment of the first aspect of the invention; and (b) a second container consisting of the ionically conductive aqueous solution of any embodiment of the composition of the first aspect of the invention.

In a fourth aspect, the present invention provides kits comprising (a) a first container comprising or consisting of the composition of any embodiment of the first aspect of the invention; and (b) a second container consisting of the ionically conductive aqueous solution of any embodiment of the first layer of the second aspect of the invention;

In a fifth aspect, the present invention provides methods for coating a hydrophobic surface, comprising coating the hydrophobic surface with a device according to the second aspect of the invention, wherein either the first layer of the device, when present, is in direct contact with the hydrophobic surface, or the second layer of the device is in direct contact with the hydrophobic surface.

### BRIEF DESCRIPTION OF THE DRAWINGS

FIG. 1 is an illustration of an experimental setup used to simulate the condensation encountered in the human eye in surgery.

### DETAILED DESCRIPTION OF THE INVENTION

In a first aspect, the present invention provides compositions, comprising a mixture of (a) a solvated viscoelastic polymeric gel, comprising a viscoelastic polymer having a molecular weight of between about 20,000 Da and about 4,000,000 Da; and (b) an ionically conductive aqueous solution; wherein the volume ratio of (a) to (b) is between 1:1 and 1:10.

The compositions of the invention can be used, for example, to prepare the devices of the second aspect of the invention, which can be usefully applied to any surface that is hydrophobic, including but not limited to silicone, hydrophobic acrylic, any form of silicon dioxide, quartz or silicon substrates used for medical device implants and surgical supplies, shunts, and tubing; and eyewear, such as sports visors, eye glasses, and goggles, having, for example high impact resistance coating such as a silicate over an underlying polycarbonate substrate. to act, for example, as an antifogging coating on a hydrophobic surface which exceptional clarity (minimum optical distortion, excellent transparency). In another example, the compositions of the invention can be used as “lubricious” agent on medical implants, shunts and surgical supplies to (a) minimize tissue trauma, (b) maximize bio-compatibility (c) increase healing by enhancing better irrigation and flow in adjacent tissue

As used herein, the term “mixture” means the two referenced elements combined, whether in emulsion form or phase-separated, such as during storage. The term emulsion is used because the mixture maintains the molecular structure of the hydrophilic viscoelastic polymeric strands, including electrolyte, water molecules, and other charged solutes attached to the polymer strand, in a free flowing solution of aqueous electrolyte.

The “ionically conductive aqueous solution” can be any suitable fluid comprising an aqueous electrolyte, such as any saline solution. In one preferred embodiment, the electrolyte in the ionically conductive aqueous comprise at least 0.03% (one ion to about 3000 water molecules) of the solution. In another preferred embodiment, the electrolyte in the ionically conductive aqueous comprise between at least 0.03% to about 0.5% of the solution; in further preferred embodiments, between at least 0.05% and about 0.4%; between at least 0.03% and about 0.3%; and at least 0.03% and about 0.2% of the solution. While not being bound by a specific mechanism of action, the inventors believe that the ionically conductive aqueous solution provides the electrical conductivity needed to generate preferred hydrophilic properties of the composition, for use in wetting a surface and/or preventing condensation droplets (see below).

In one preferred embodiment, the ionically conductive aqueous solution comprises or consists of a solution with the following characteristics (Table 1):

TABLE 1

| Component | | |
|---|---|---|
| Na⁺ | 144 to 164 | meq/L |
| K⁺ | 2 to 10.1 | meq/L |
| Ca²⁺ | 1 to 3.3 | meq/L |
| Mg²⁺ | 0.0 to 1.5 | meq/L |
| Cl⁻ | 128 to 177 | meq/L |
| HCO₃⁻ | 0.0 to 20 | meq/L |
| Phosphate | 0.0 to 3.0 | meq/L |
| Lactate | 0.0 to 8.0 | meq/L |
| Glucose | 0.0 to 5.0 | meq/L |
| Ascorbate | 0.0 to 2.0 | meq/L |
| Glutathione | 0.0 to 0.3 | meq/L |
| Citrate | 0.0 to 6.0 | meq/L |
| Acetate | 0.0 to 3.0 | meq/L |
| pH | 7.1 to 7.7 | |
| Osmolality | 250 to 350 | mOsmoles/L |

Thus, in one preferred embodiment, the ionically conductive aqueous solution comprises or consists of Na⁺, K⁺, Ca²⁺, Mg²⁺, and Cl⁻ within the range noted in Table 1, and has a pH and osmolality within the ranges noted in Table 1. The other components may be optionally added either individually or in any combination to prepare the final ionically conductive aqueous solution.

In another preferred embodiment, an ionically conductive aqueous solution approximating (+/−10% for each component) the composition of human vitreous humor, approximating the composition of human aqueous humor, or any saline solution used in eye surgery or therapy, can be used (Table 2). In further preferred embodiments, the ionically conductive aqueous solution is a solution comprising or consisting essentially of or consisting of components +/−9%, 8%, 7%, 6%, 5%, 4%, 3%, 2%, 1%, or identical to those listed in a solution listed in Table 2.

TABLE 2

| | | | | BSS | |
|---|---|---|---|---|---|
| | | | Hartman's | PLUS ® | BSS ® |
| | Human | Human | Lactated | Intraocular | Intraocular |
| Ingredient | Aqueous | Vitreous | Ringer's | Irrigating | Irrigating |
| in meq/L | Humor | Humor | Solution | Solution | Solution |
| Sodium | 162.9 | 144 | 102 | 160.0 | 155.7 |
| Potassium | 2.2-3.9 | 5.5 | 4 | 5.0 | 10.1 |
| Calcium | 1.8 | 1.6 | 3 | 1.0 | 3.3 |
| Magnesium | 1.1 | 1.3 | — | 1.0 | 1.5 |
| Chloride | 131.6 | 177.0 | — | 130.0 | 128.9 |
| Bicarbonate | 20.15 | 15.0 | — | 25.0 | — |
| Phosphate | 0.62 | 0.4 | — | 3.0 | — |
| Lactate | 2.5 | 7.8 | 28 | — | — |
| Glucose | 2.7-3.7 | 3.4 | — | 5.0 | — |
| Ascorbate | 1.06 | 2.0 | — | — | — |
| Glutathione | 0.0019 | — | — | 0.3 | — |
| Citrate | — | — | — | — | 5.8 |
| Acetate | — | — | — | — | 28.6 |
| pH | 7.38 | — | 6.0-7.2 | 7.4 | 7.6 |
| Osmolatity | 304 | — | 277 | 305 | 298 |
| (mOsm) | | | | | |

Various further preferred embodiments for the ionically conductive aqueous solution are those that approximate the compositions of body fluid compartments, for example, those with the ions and protein additives as listed in Table 3 below. The extracellular fluid compartment (ECF) is composed of the blood/plasma compartment and the interstitial fluid compartment.

TABLE 3

| Fluid Compartment | Component | Composition elements |
|---|---|---|
| Extracellular | Blood/plasma | Na⁺, K⁺, Ca²⁺, Mg²⁺, |
| | | HCO₃⁻, Cl⁻, HPO₄²⁻, SO₄²⁻, |
| | | organic acids, protein, |
| | | non-electrolytes |
| | Interstitial | Na⁺, K⁺, Ca²⁺, Mg²⁺, |
| | | HCO₃⁻, Cl⁻, HPO₄²⁻, SO₄²⁻, |
| | | organic acids, protein, |
| | | non-electrolytes |
| Intracellular | Cells | K⁺, Na⁺, Mg²⁺, HCO₃⁻, |
| | | Cl⁻, PO₄³⁻, SO₄²⁻, |

In one non-limiting preferred embodiment, the ionically conductive aqueous solution is normal saline (NS). Normal saline (NS) is the commonly-used term for a solution of 0.91% w/v of NaCl, about 300 mOsm/L; it is also known as physiological saline or isotonic saline. The solution is 9 grams of sodium chloride (NaCl) dissolved in 1 liter of water, and contains 154 mEq/L of Na⁺ and Cl⁻. It has a slightly higher degree of osmolarity (i.e. more solute per liter) than blood. Nonetheless, the osmolarity of normal saline is a close approximation to the osmolarity of NaCl in blood. Thus, in this embodiment, an ionically conductive aqueous solution of approximately 0.154 M salt is used.

Commercially available examples of ionically conductive aqueous solutions are BSS PLUS® intraocular irrigating solution, include, but are not limited to, balanced saline solution (BSS) from Akorn (IL) and BSS® intraocular irrigating solution (Alcon Laboratories, Inc., Fort Worth, Tex.). For applications in human device implants and medical implants, normal saline is a preferred electrolyte of choice. For application to the most delicate tissues and to minimize inflammation and promote tissue healing after implantation, balanced saline solution is a preferred ionically conductive aqueous solution.

The composition also comprises a solvated viscoelastic polymeric gel, comprising a viscoelastic polymer having a molecular weight of between about 20,000 Da and about 4,000,000 Da. As used herein, the term “viscoelastic” means that the component exhibits both viscous and elastic properties when undergoing deformation.

The preferred viscoelastic polymeric gel is solvated, which means that the polymer chains are surrounded by an essentially continuous molecular tube made of water molecules whose dipoles are aligned to form a solvation “cage” around the polymer chains to foam gel “strands” (like strands of pearls where the polymer is the thread and the water molecules are the surrounding pearls). As used herein, ‘solvated” means the water molecules associate with polymer chain by electrostatic dipole-dipole interactions between the water molecules and polymer components. Solvation of the viscoelastic polymer allows for the presence of ions in the gel, which can enhance conduction and electrostatic interaction along the polymer chains.

In preparing the solvated viscoelastic polymeric gel of the invention, the polymer chains are preferably initially well-hydrated; preferably, the polymer is at least 60%, 70%, 80%, 90%, or fully hydrated; or between 60-100%, 70-100%, 80-100%, or 90-100%. Solvation can be measured during preparation of the gel. For example, the polymer can be a powder measured by weight, and the solvent (e.g., water) can be measured by volume.

In other preferred embodiments, the viscoelastic polymer can have a molecular weight of about 40,000 Da to about 4,000,000 Da; preferably between 30,000 Da and 2,000,000 Da, and even more preferably between 40,000 Da and 1,000,000 Da. Lower molecular weights are preferred, such as about 20,000 Da to about 1,000,000 Da, as gravity can cause the polymer to delaminate and detach from a surface on which the composition may be positioned.

As used herein, the “molecular weight” of a polymer refers to the weight-averaged molecular weight of the referenced polymer.

In various further preferred embodiments, the viscoelastic polymer has a molecular weight of between about 40,000 Daltons (Da) and about 500,000 Da; about 40,000 Da and about 250,000 Da; about 40,000 Da and about 150,000 Da; about 40,000 Da and about 100,000 Da; about 40,000 Da and about 80,000 Da; about 50,000 Da and about 500,000 Da; about 50,000 Da and about 250,000 Da; about 50,000 Da and about 120,000 Da; about 50,000 Da and about 110,000 Da; about 50,000 Da and about 100,000 Da; about 50,000 Da and about 90,000 Da; about 50,000 Da and about 80,000 Da; about 60,000 Da and about 500,000 Da; about 60,000 Da and about 250,000 Da; about 60,000 Da and about 120,000 Da; about 60,000 Da and about 110,000 Da; about 60,000 Da and about 100,000 Da; about 60,000 Da and about 90,000 Da; about 60,000 Da and about 80,000 Da; about 70,000 Da and about 500,000 Da; about 70,000 Da and about 250,000 Da; about 70,000 Da and about 120,000 Da; about 70,000 Da and about 110,000 Da; about 70,000 Da and about 100,000 Da; about 70,000 Da and about 90,000 Da; about 70,000 Da and about 80,000 Da; about 80,000 Da and about 500,000 Da; about 80,000 Da and about 250,000 Da; about 80,000 Da and about 120,000 Da; about 80,000 Da and about 110,000 Da; and about 80,000 Da and about 100,000 Da.

Any physiologically compatible viscoelastic polymer (which can comprise a combination of polymers), capable of forming a solvated gel, and otherwise meeting the requirements recited herein can be used in the present invention. In a preferred embodiment, the viscoelastic polymer is hydrophilic, to promote solvation of the polymer and to enhance conduction and electrostatic interaction along the polymer chains (infra). As will be understood by those of skill in the art, and based on the teachings herein, the molecular structure, molecular weight, and concentration of the polymer can determine the characteristics of the resulting polymeric gel.

In one preferred embodiment, the solvated viscoelastic polymeric gel is homogenous. As used herein, the term “homogeneous” is defined as a single polymer type in the gel. In another preferred embodiment, the solvated viscoelastic polymer comprises a combination of two or more polymers.

A wide spectrum of viscoelastic polymers can be used herein. At the one end of the spectrum are more fluidic polymers, such as hydroxypropylmethylcellulose (HPMC), which are able to coat a surface. At the other end of the spectrum are more viscous polymers, such as hyaluronic acid. HPMC-based gels can coat (“dispersive”) and protect endothelial (skin) tissues from damage, and are much cheaper, while hyaluronic acid-based gels are designed to protect the eye chamber from collapse and are mechanically more cohesive. Similar viscoelastics can be made out of sodium hyaluronate and chondroitin sulfate.

Polymers used can range from hyaluraonic acid (which can be extracted from animal tissue in various polymeric lengths, forms, purity and concentration) to plant-based cellulose in various polymeric lengths, forms, purity and concentration). Thus, for example on-limiting, exemplary viscoelastic polymers that can be used in the compositions of the invention include, but are not limited to hyaluronic acid and similar molecules (basic monomer C₃₃H₅₄N₂O₂₃, 846.8 grams/mol), which is the polymer forming the eye vitreous humor; hydroxypropylmethylcellulose (HPMC) (basic monomer C₃₂H₆₀O₁₉, 748.8 grams/mole) in the stoichiometry C₆H₇O₂(OH)ₓ(OCH₃)_{y}(OC₃H₇)_{z} ((x+y+z=3); hydroxyethylmethylcellulose and mixtures thereof, or various combinations of shorter polymeric segments (oligomers) thereof (also referred to as “intermediate formulations”).

In a further preferred embodiment, the viscoelastic polymer comprises short lateral side groups which ensure proper meshing of the chains when the gel is mixed. Three connecting side groups are typically sufficient for meshing of the chains. As used herein, “short side groups” means that the side arms of the monomer are shorter than the monomer itself.

In HPMC, the hydroxypropyl side groups can mechanically interact by tangling with surrounding molecules. A completely linear polymer would not work as well to create an open mesh of fully solvated polymer strands. Hydroxy side chains of HPMC also favor solvation and hydrophilic properties making this polymer more cohesive and more likely to interact with nearby strands

As used herein, “proper meshing” means that the fully solvated polymer strands, when emulsified in the ionically conductive aqueous solution, still mechanically and electrostatically interact with one another to faun an open network of connected polymeric strands in the manner of a weft in an open weave fabric. New water molecules arising from condensation can freely flow through the holes of the open weave. However, the strands also provide a continuous and stable network where ionic conduction can occur while maintaining hydrophilic properties without any dilution effect from the accumulation of pure condensed water. This stable, hydrophilic open mesh of hydrated polymer strands helps maintain the elimination of condensation (fogging) for extended periods of times, such as during eye surgery where moisture distilled from the tissue can condense on a silicone IOL. The dilution of the emulsion also ensures minimum optical distortion and optimal clarity, which cannot be achieved with only a viscoelastic polymeric gel.

In a further preferred embodiment, the viscoelastic polymer comprises hydroxy side groups which enhance solvation and decrease agglomeration as seen in sodium hyaluronate. Akin to human vitreous humor, hyaluronates are more cohesive and viscous. Hydroxy termination of the monomer enhances polarization and attraction with water molecules, which enhances solvation and electrostatic interaction leading to the desired hydrophilic properties. Simple monomer side chains, such as —CH₂—OH—CH₃ and hydroxy groups, are less likely to shield or interfere with solvation than more complex ones hydroxypropyl groups (—CH₂C(H)(OH)CH₃). HPMC is dominated by such simple monomer side chains, while the structure of hyaluronic acid is much less so, making it more difficult to fully solvate.

HPMC is a preferred embodiment compared to hyaluronates as the lower molecular weight chains of HPMC form less viscous polymer gels than those formed from hyaluronic acid. The ratio of H to C is 1.875 (60/32), and H to O is 3.15 (60/19) within the hydroxypropylmethylcellulose (HPMC) monomers is more favorable than the H/C ratio of 2.35 (54/32) and the H/O ratio of 2.35 (54/23) in hyaluronate monomers because the former side groups are shorter (contain fewer atoms) and more likely to hydrate.

The solvated viscoelastic polymeric gel may be prepared directly from dry powdered viscoelastic polymer by hydrating the dry polymer by addition to water (e.g, deionized water or 18 MΩ deionized water) or an aqueous electrolyte. The aqueous electrolyte can have a composition as described previously for the ionically conductive aqueous solution of the composition of the invention, however, it does not have to be identical.

In one preferred embodiment, a solvated viscoelastic polymeric gel can be formed by hydrating a dry polymer powder in an aqueous electrolyte of composition similar, but not necessarily identical to the ionically conductive aqueous solution of the composition. If such polymers are put directly into saline, they may agglomerate, a less preferable embodiment. In another embodiment, a solvated viscoelastic polymeric gel can be formed by hydrating a dry polymer powder in deionized water (e.g., 18 MΩ deionized water). Separate preparation of the solvated viscoelastic polymeric gel permits formation of a well-hydrated autonomous gel which facilitates formation of a lightweight and open mesh in the next step. Once the solvated viscoelastic polymeric gel is prepared, it can be emulsified in the ionically conductive aqueous solution in the desired ratio, as discussed below.

In one preferred embodiment, the solvated viscoelastic polymeric gel can have a polymer concentration between about 1% and about 10% by weight. In various preferred embodiments, the polymer concentration is between about 1% and about 9%; or about 1% and about 5%; or about 1% and about 7%; or about 1% and about 6%; or about 1% and about 5%; or about 1% and about 4%; or about 1% and about 3%; or about 1% and about 2%; or about 1.5% and about 10%; or about L5% and about 9%; or about 1.5% and about 8%; or about 1.5% and about 7%; or about 1.5% and about 6%; or about 1.5% and about 5%; or about 1.5% and about 4%; or about 1.5% and about 3%; or about 2% and about 10%; or about 2% and about 9%; or about 2% and about 8%; or about 2% and about 7%; or about 2% and about 6%; or about 2% and about 5%; or about 2% and about 4%; or about 2% and about 3%.

In another preferred embodiment, a commercial viscoelastic polymeric gel can be used for preparing the solvated viscoelastic polymeric gels of the invention. Non-limiting examples of commercial viscoelastic polymeric gels that can be adapted for use, for example, by the proper dilution, in the present invention include any gel that has been FDA approved for eye surgery, including, but not limited to:
(1) OcuCoat® (Bausch & Lomb) comprises 2% 80 KDa Hydroxypropyl methylcellulose (20 mg/mL), sodium chloride (0.49%), potassium chloride (0.075%), calcium chloride (0.048%), magnesium chloride (0.03%), sodium acetate (0.39%), sodium citrate (0.17%), remainder water, the composition having a viscosity of 4000±1500 cst;
(2) Viscoat® (Alcon Laboratories) comprises a buffered solution of 3% 500,000 Da sodium hyaluronate (30 mg/mL) and 4% 22.5 KDa chondroitin sulfate, and has a viscosity of about 40000±20000 cps and a pH of 7.2±0.2;
(3) Healon® (Abbott Medical Optics) comprises 1% 4.0 MDa sodium hyaluronate (10 mg/mL) in a sodium/chloride/phosphate buffer , and has a viscosity of about 300,000 mPas and a pH of about 7.0-7.51;
(4) DuoVisc® (Alcon Laboratories) is a combination of Viscoat® and ProVisc® at varying ratios. Both Viscoat®, as listed above, and Provisc®, are sodium hyaluronate solutions a sodium/chloride/phosphate buffer;
(5) Amvisc® (Bausch & Lomb), comprises a solution of 1.2% 2.0 MDa sodium hyaluronate (16 mg/mL) in a physiological sodium chloride phosphate buffer solution (pH 6.8-7.6), and having a viscosity of about 132,000 cP at 25° C. and an osmolality of approximately 340 mOsmol;
(6) Amvisc® PLUS (Bausch & Lomb), comprises a solution of 1.6% 1.5 MDa sodium hyaluronate (16 mg/mL) in a physiological sodium chloride phosphate buffer solution (pH 6.8-7.6), and having a viscosity of about 132,000 cP at 25° C. and an osmolality of approximately 340 mOsmol;
(7) BioLon® (Bio-Technology General (Israel) Ltd.) comprises a solution of 1.0% 3 MDa sodium hyaluronate;
(8) Cellugel® (Alcon Labs) comprises a solution of 2.0% 300 KDa HPMC;
(9) CoEase® (Advanced Medical Optics, Inc.) comprises a solution of 1.2% 1 MDa sodium hyaluronate;
(10) EyeVisc™ (Biotech Visioncare) comprises a solution of 2.0% HPMC;
(11) EyeVisc™ Plus (Biotech Visioncare) comprises a solution of 2.0% HPMC;
(12) EyeVisc™ SH (Biotech Visioncare) comprises a solution of 1.4% sodium hyaluronate;
(13) Healon® GV (Abbott Medical Optics) comprises a solution of 1.4% 5 MDa sodium hyaluronate;
(14) LensVisc™ MC (LensTec) comprises a solution of 2.0% 86 KDa HPMC;
(15) LensVisc™ HA (LensTec) comprises a solution of 2.0% 2.3 MDa sodium hyaluronate;
(16) Occu-Lon™ cornprises a solution of 1.5% 2 MDa sodium hyaluronate;
(17) ShellGel™ (Cytosol Ophthalmics, Inc) comprises a solution of 1.2% sodium hyaluronate;
(18) STAARVisc® II (STAAR Surgical Company) comprises a solution of 1.2% sodium hyaluronate;
(19) UniVisc™ (CIBA Vision) comprises a solution of 1.0% 3 MDa sodium hyaluronate; and
(20) Vitrax (Abbott Medical Optics) comprises a solution of 3.0% 500 KDa sodium hyaluronate.

See, for example, Rice, D. J et al., C. J., Ophthalmologic Drug Guide, Springer: New York, 2007, pages 90-91, which is hereby incorporated by reference.

In certain preferred embodiments, the viscoelastic polymeric gel is Viscoat®, Amvisc®, or Duovisc®.

When a commercial viscoelastic polymeric gel is used for preparing a solvated viscoelastic polymeric gel of the compositions of the invention, and when such commercial viscoelastic polymeric gel is solvated as provided, it may be used directly for mixing with an ionically conductive aqueous solution. For example, a preferred embodiment of a viscoelastic polymeric gel is Healon®, FDA approved for eye surgery, which comprises fully solvated hyaluronic acid (10 mg/mL).

However, when such commercial gels are not fully solvated, the gel can be combined with a suitable amount of an aqueous medium to fully solvate the polymer in the commercial viscoelastic polymeric gel. For example, Healon® 5 (23 mg/mL) is not fully solvated. When Healon® 5 is used, additional aqueous solution can be added to fully solvate the polymer chain (for example, 2.3 parts of an aqueous solution to 1 part of Healon 5). The volume ratio of Healon 5 to aqueous solution allows for complete solvation of the viscoelastic polymeric gel.

In a preferred embodiment, the viscoelastic polymer comprises HPMC (e.g., OcuCoat®). In other embodiments, any of the preceding commercially available viscoelastic polymers may be used with appropriate dilution (see below), or degraded into shorter polymer chains to meet the molecular weight limitations recited herein for the viscoelastic polymer.

As will be understood by those of skill in the art, and based on the disclosure herein, the viscoelastic polymer concentration can be adapted to produce a solvated viscoelastic polymeric gel having the desired viscosity. The desired viscosity of the gel should (1) allow for formation of a homogeneous mixture without agglomeration; and (2) be high enough to maintain the device of the invention (see below) as a stable sheet on a surface (i.e., silicone) despite gravity.

In a further preferred embodiment, the viscosity of the solvated viscoelastic polymeric gel is between about 50 cP and about 5000 cP. In further preferred embodiments, the viscosity of the solvated viscoelastic polymeric gel is between about 50 cP and about 4500 cP; or about 50 cP and about 4000 cP; or about 100 cP and about 5000 cP; or about 100 cP and about 4000 cP; or about 100 cP and about 3000 cP; or about 200 cP and about 3000 cP; or about 100 cP and about 2000 cP; or about 200 cP and about 2000 cP; or about 100 cP and about 1500 cP; or about 200 cP and about 1500 cP; or about 100 cP and about 1000 cP; or about 100 cP and about 1000 cP.

In one non-limiting example, a tenfold decrease in polymer molecular weight can result in a viscosity decrease of over 1000 times. For example, a 20,000 Da cellulosic gel could be diluted by only 8% in water by weight. A 20,000 Da polymer could have a viscosity as low as few hundreds centipoise (cP); increasing the polymer concentration could provide an increase of viscosity by about two order of magnitude. Then, the viscosity could be in the tens of thousands of cP, for example, in the range of about 20,000 cP, and require appropriate dilution in the gel (e.g., with water or an ionically conductive aqueous solution) to achieve a preferred viscosity.

In a further preferred embodiment, hydroxypropylmethylcellulose (HPMC) of molecular weight 86,000 Da is used at 2% by weight in saline, resulting in solvated viscoelastic polymeric gel having a viscosity of about 4000 cP.

In a further preferred embodiment, a 2.5% HPMC (86,000 Da) gel by weight in saline exhibits a viscosity of 15,000 cP. Although a higher viscosity than preferred for forming the composition, higher concentration gels have value, for example, by extending the time to condensation, when used in the devices and methods of the invention (see below). In a further embodiment, 120,000 Da HPMC, a vitreous substitute, can be hydrated in a gel to a concentration of 2% by weight in saline, prior to mixture with an ionically conductive aqueous solution to yield a matching viscosity of about 15,000 cP.

In another preferred embodiment, the solvated viscoelastic polymer gel can be a coherent (i.e., does not break apart) and well-hydrated gel. Such preferred gels can yield a hydrophilic viscoelastic gel having sufficient viscosity (4000 cP) to form an open hydrophilic molecular mesh of lower viscosity which can be used as the second layer component in the devices of the invention (infra).

In a further preferred embodiment, the solvated viscoelastic polymeric gel is prepared such that there are enough water molecules in the gel to create well-hydrated polymer chains with a “cage” of water dipoles surrounding the chain in an essentially complete first solvation radius, with the electrolytes in the ionically conductive aqueous solution, when used to prepare the gel, serving as electrostatic attractors for the next step when the gel is mixed in an ionically conductive aqueous solution in the desired ratio. For example, well-hydrated polymeric chains can include about 20 to about 100 water molecules per monomer along the polymeric chain; preferably between 30 and about 100; or about 40 and about 100; or about 50 and about 100; or about 60 and about 100; or about 70 and about 100; or about 80 and about 100 water molecules per monomer along the polymeric chains. The number of water molecules per monomer can be derived from the monomer length and the first, second and third solvation radii.

In a further preferred embodiment, a solvated 2 wt % viscoelastic HPMC gel is prepared so that there are at least 80-100 molecules per monomer along the polymer chains. For 80,000 Da HPMC, a 2% gel in saline has about 200,000 water molecules for each polymeric chain. Such a polymeric chain has about one thousand monomers per chain, on average, corresponding to about 200 water molecules around each monomer. Such is sufficient to build a complete first solvation radius; the extra water molecules avoid any “bare”, unhydrated CH-based molecular terminations and initiate the second solvation radius. In other embodiments of this example, hydrating between 60-100%, 70-100%, 80-100%, or 90-100% of the CH-based molecular terminations are suitable. That is, main polymeric chain and its side groups have about the same hydration requirements.

A composition of the invention can be formed by an at least two step process. First, a viscoelastic polymer is fully solvated to form the solvated viscoelastic polymeric gel by addition of an aqueous solution (e.g., water or an ionically conductive aqueous solution). Then, an amount of an ionically conductive aqueous solution is added to dilute the solvated viscoelastic polymeric gel into a composition of the invention.

The volume ratio of the solvated viscoelastic polymeric gel to the ionically conductive aqueous solution is between 1:1 and 1:10 depending on the molecular weight of the polymer in the solvated gel. For lower molecular weight polymers, such as HPMC, a ratio of 1:3 or 1:5 works well. For higher molecular weight polymers, such as hyaluronic acid, it is best to emulsify the solvated polymeric gel at a higher dilution, in 1:10 ratio. In various preferred embodiments the ratio is about 1:1, 1:2, 1:3, 1:4, 1:5, 1:6, 1:7, 1:8, 1:9, 1:10, 3:10, 7:10, 9:10, 2:9, 2:7, 2:5, 2:3, 3:8, 3:7, 3:5, 3:4, 4:9, 4:7, 5:9, 5:8, 5:7, 5:6, 6:7, 7:9, 7:8, and about 8:9. In a further preferred embodiment, the ratio is between 1:3 and 1:5.

In certain preferred embodiments, the composition of the invention comprises between about 0.0003 wt % and about 10 wt % viscoelastic polymer. For example, the composition can contain between about 0.0003 wt % and about 5.0 wt %; or between about 0.0003 wt % and about 3.0 wt %; or between about 0.0003 wt % and about 2.0 wt %; or between about 0.0003 wt % and about 1.0 wt %; or between about 0.0005 wt % and about 10 wt %; or between about 0.0005 wt % and about 5.0 wt %; or between about 0.0005 wt % and about 3.0 wt %; or between about 0.0005 wt % and about 2.0 wt %; or between about 0.0005 wt % and about 1.0 wt %; between about 0.001 wt % and about 10 wt %; or between about 0.001 wt % and about 5.0 wt %; or between about 0.001 wt % and about 3.0 wt %; or between about 0.001 wt % and about 2.0 wt %; or between about 0.001 wt % and about 1.0 wt %; between about 0.01 wt % and about 10 wt %; or between about 0.01 wt % and about 5.0 wt %; or between about 0.01 wt % and about 3.0 wt %; or between about 0.01 wt % and about 2.0 wt %; or between about 0.01 wt % and about 1.0 wt %; between about 0.1 wt % and about 10 wt %; or between about 0.1 wt % and about 5.0 wt %; or between about 0.1 wt % and about 3.0 wt %; or between about 0.1 wt % and about 2.0 wt %; or between about 0.1 wt % and about 1.0 wt % viscoelastic polymer.

When the solvated viscoelastic polymeric gel is mixed with an ionically conductive aqueous solution to form a composition of the invention, water molecules can easily surround the well-hydrated polymer chains. Free electrostatic interactions between the electrolytes and the freely flowing water molecules from the added well-hydrated polymer enables adhesion of the resulting emulsion with, for example, a compatible saline film, thus forming a bilayer embodiment of the device of the invention (as discussed below).

Alternatively, a composition of the invention can be foimed by direct hydration of a viscoelastic polymer in the ionically conductive aqueous solution, provided that the viscoelastic polymer is well-hydrated and does not aggregate. Such directly prepared compositions should conform to the dilution requirements as described above.

The volume of the resulting composition can be any volume suitable for a particular use. For delivery, the only limitation on the volume is to provide enough to create a continuous film that is at least one molecular layer thick on a surface to be coated. Since the composition comprises macromolecules, the thickness is typically more than 1 nm. In a non-limiting embodiment, a droplet of about 0.5 μL or more of the composition on a lens is suitable to cover a 5 or 6 mm lens.

All of the embodiments of the composition of the invention can be used together in any combination except where the context clearly dictates otherwise.

In a second aspect, the present invention provides a device, comprising or consisting of (a) an optional first layer comprising or consisting of an ionically conductive aqueous solution; and (b) a second layer comprising or consisting of the composition of any embodiment of the first aspect of the invention in emulsion form; wherein, when the first layer is present, the first layer and the second layer are in direct contact.

The adhesion of the first and/or second layers to the surface is ensured by the water component of the emulsion. Cohesion of the applied layer sets a mininum amount of water in the emulsion and the hydration requirement of the polymer. For example, if an emulsion is not used and a pure gel is applied, it can delaminate from the surface. During application, both the first and second layers may be merged as a single layer in direct contact with the surface since the matrix component of the emulsion is water. For example, a properly hydrated polymer in a properly prepared solution, each as described in any of the preceding embodiments of the compositions of the invention, can wet the surface uniformly, avoiding direct contact of the gel with the surface, and maintaining a flowing free water layer between the surface and the emulsion while the hydrated polymeric chain are found in the water matrix of the applied emulsion.

As noted, all embodiments of the composition of the first aspect, as well as all embodiments of the solvated viscoelastic polymeric gel component and ionically conductive aqueous solution components of the compositions can be used in the device of this second aspect.

In one preferred embodiment of the second aspect, the device has a density between about 0.5 monomers/nm² and about 2000 monomers/nm²; or between about 0.5 monomers/nm² and about 1750 monomers/nm²; between about 1.0 monomers/nm² and about 1750 monomers/nm²; between about 10 monomers/nm² and about 1750 monomers/nm²; between about 100 monomers/nm² and about 1750 monomers/nm².

In another preferred embodiment of the second aspect, the device has an areal density of between about 10¹⁸ atom/cm² and about 10¹⁹atom/cm² as measured by He⁺⁺ Rutherford backscattering spectrometry (RBS).

The present invention resolves condensation problems on silicone-based and hydrophobic acrylic IOLs, allowing more patients access to lenses with superior optical properties and stability as compared to the older, opacifying acrylic and PMMA IOLs. The devices of the invention can be applied both prior to surgery ex situ and during surgery in situ on already implanted silicone lenses. Furthermore, the devices of the invention can be applied to any surface that is hydrophobic, including but not limited to silicone, hydrophobic acrylic, any form of silicon dioxide, quartz or silicon substrates used for medical device implants and surgical supplies, shunts, and tubing; and eyewear, such as sports visors, eye glasses, and goggles, having, for example high impact resistance coating such as a silicate over an underlying polycarbonate substrate.

The device can comprise either a bilayer consisting of two sheets, or a single layer of the composition of the invention, each forming a continuous, planar coating. The device can (1) act as an antifogging coating on a hydrophobic surface which exceptional clarity (minimum optical distortion, excellent transparency); and (2) act as “lubricious” agent on medical implants, shunts and surgical supplies to (a) minimize tissue trauma, (b) maximize bio-compatibility (c) increase healing by enhancing better irrigation and flow in adjacent tissue. The layer(s) of the device are compatible with a continuous flow of distilled water vapor in unlimited amounts, since they resist condensation as demonstrated herein.

In one preferred embodiment, the devices of the present invention are designed to adapt to the unique electronic properties and concomitant adhesive response of silicone. Silicone is a composite material made of two molecular components, and consequently exhibits two different, parallel, antagonistic electronic and adhesive properties and responses upon exposure to water molecules. The dual response of silicone initially causes water vapor to condense in droplets rather than wet the surface in a uniform film. The strong hydrophilic adhesion of these droplets on an initially hydrophobic silicone makes the intraocular lenses (IOLs) opaque.

In one preferred embodiment, a device of the invention is disposed on a hydrophobic surface, wherein the first layer and second layers comprise a combined single continuous layer that is in direct contact with the hydrophobic surface (i.e., the second layer, as described herein, is in direct contact with the hydrophobic surface).

In a further preferred embodiment, the devices of the invention can be built as two separate molecular layers. When built as two layers, each can overcomes the response of one of the two components of silicone. The first layer can act as a wetting agent and also provides an electrically active bonding layer made of an aqueous electrolyte, while the second layer preferably is a hydrophilic viscoelastic polymeric emulsion, made out of fully solvated polymeric strands in an electrolyte electrically and chemically similar in composition to the first (bonding) layer. Active electrical conduction is accomplished via the electrolyte in both layers, and mechanical shielding of the bonding layer is accomplished via the viscoelastic properties of the electrically conductive emulsion.

When present, the first layer can ensure that the hydrophobic surface is thoroughly wetted by using an ionically conductive aqueous solution that is electrically polarizable. For example, small electrolytes, such as Na+ have an edge over larger molecules because they are among the most mobile species in nature in a solid or a liquid. Thus, such a choice of electrolyte enables them to move freely in the aqueous solution, unlike the fixed charges in, for example, silicon oxide molecules forming silicone lens materials or in the long polymeric chains of most viscoelastic gels. Electrolytes in ionically conductive aqueous solution act as free electrical carriers that have a fast polarization response when in contact with any medium. Polarization in the aqueous solution means that charges of opposite sign arrange themselves by electrical attraction to the charges that are fixed in the dipoles forming the silicone materials. These properties allow the first layer to act as a sticking or bonding layer for the second layer as follows (discussed in the non-limiting context of an IOL).

The great mobility of electrolytes in the aqueous solution allows them to arrange themselves very quickly by their attraction between negative and positive charges found within a silicone IOL in the form of fixed, immobile silicon dioxide dipoles chains. These chains form the backbone of the composite silicone molecules, which are terminated with less polarized hydrocarbons.

Given the fast response from the electrolytes, water molecules in the aqueous solution can also more easily interact electrically with the free electrolytes than between each other and thus arrange themselves as a uniform aqueous film on the silicone surface rather than as opacifying droplets. The electrical attraction between mobile charges and the silicone strong molecular dipole on the one end, and water dipoles on the other end ensures a uniform wetting aqueous film instead of nucleating condensation droplets. This electrostatic bond is very significant and gives the electrolyte-rich aqueous film its mechanical stability.

However, use of only a first electrolyte-rich aqueous film does not avoid condensation on the IOL. In the same way, covering the IOLs with pure gels fails because their long molecular structure do not allow for the water distillate to electrically interact with them at them at the molecular level and stick in a uniform film instead of nucleating as discrete droplets. That is, wetting the silicone lens with only a saline solution or only a gel does not prevent condensation.

In a preferred embodiment, thorough rinsing, purity of the ionically conductive aqueous solution and the tools used to deliver it, and uniformity of the ionically conductive aqueous solution, easily achieved in a sterile environment, are employed to ensure uniformity of the first layer. Purity of the ionically conductive solution should corresponds to at least about one MΩ of water resistivity (typical of commercial grade distilled water) prior to adding the physiological salts required for saline.

As described in detail in the first aspect of the invention, the composition that form is the second layer, or a single layer of the device, can be a composite, electrolyte rich layer that is an emulsion of an ionically conductive aqueous solution and a solvated viscoelastic polymeric gel.

A condensed water vapor yields a pure water distillate, thus devoid of electrolytes, even when originating from the human body during surgery. Water condensation on silicone during surgery is severe because the condensed water is a pure distillate. However there is natural chemical affinity between the water molecules in the distillate condensing on the posterior surface of the IOLs and an aqueous film, such that direct condensation on an aqueous film dilutes quickly the electrolytes distribution in the film. This negates its effects and droplets quickly appear (in 10 to 15 seconds)

The unique approach in the devices of the present invention to build the second layer, or the combined single layer, is to combine the electrolytic conduction of an ionically conductive aqueous solution with a surgically compatible, optically transparent medium with radically different molecular structure, into a uniform emulsion that is not only a gel, nor an aqueous solution.

In use, the first and second layers can be disposed on a hydrophobic substrate as continuous films. In a preferred embodiment, the minimum thickness of first and second layers, or a combined single layer, are each at least 3 to 4 times the roughness of the hydrophobic surface to be coated. If the layer is too hick, it can create a droplet with optical distortion and risk of delamination (dripping down) and loss of the film from the surface.

Thus, in one non-limiting embodiment, for a smooth hydrophobic surface with a roughness of 10 nm, the minimum thickness of the first, second, or combined single layers each would be at least about 30 to 40 nm; for hydrophobic surface with a roughness of 100 nm, about 300 to 400 nm. In further preferred embodiments, the thickness of each layer is between about 30 nm and about 2 mm; or about 50 nm and about 1 mm; or about 75 nm and about 750 μm; or about 100 nm and about 500 μm; or about 250 nm and about 400 μm; or about 500 nm and about 500 μm; or about 1 μm and about 500 μm; or about 1 μm and about 400 μm; or about 10 μm and about 400 μm; or about 100 μm and about 500 μm; or about 100 μm and about 400 μm; or about 200 μm and about 500 μm; or about 200 μm and about 400

Thus, in a further preferred embodiment, a device of the invention is disposed on a hydrophobic surface, wherein the first layer is in direct contact with the hydrophobic surface, and wherein each of the first layer and the second layer are present as a continuous layer.

The hydrophobic surface may be any on which the device of the invention can be usefully applied as a coating, including but not limited to silicone, hydrophobic acrylic, any form of silicon dioxide, quartz or silicon substrates used for medical device implants and surgical supplies, shunts, and tubing; and eyewear, such as sports visors, eye glasses, and goggles, having, for example high impact resistance coating such as a silicate over an underlying polycarbonate substrate.

In various preferred embodiments, the volume of each layer can be any suitable for a given use. Similarly, any ratio of volumes of the different layers can be used as suitable for a given purpose; in one preferred embodiment, identical volumes are used. In another preferred embodiment, the volume of the aqueous layer does not result in a thicker layer than the emulsion.

For delivery, the only limitation on the volume of the second layer and/or combined single layer is to provide enough to create a film that is at least one molecule thick (more than a 1 nm, based on the polymer in the composition). Typically, a droplet of about 0.5 μL to about 1.0 μL for both layers on a lens will be a minimum to cover a 5 mm or 6 mm lens. In various preferred embodiments, the volume of the second layer ranges between 0.5 mL and 100 mL, depending on the area to be covered.

In one preferred embodiment, the second layer and/or combined single layer has a volume of about 0.5 μL to about 1.0 or more. Below such volumes, viscosity may limit the ability to control the composition ratio. In one embodiment, combining 0.5 mL of the viscoelastic polymeric gel with 0.5 mL of the ionically conductive aqueous solution (1:1) yields an emulsion volume of 1 mL for mixing and delivery of the second layer and/or combined single layer on the lens. In a further embodiment, an emulsion of 10 mL is used for preparation in the operating room with using 0.5 mL to 1 mL for delivery of the lens via syringe.

The ionically conductive aqueous solution of the first and second layers of the composition may be the same or similar. Small differences (+/−20%) in electrolyte composition are suitable so long as the two layers have good ionic conductivity. Preferred osmolalities are as described above in the first aspect of the invention.

In a third aspect, the present invention provides kits comprising: (a) a first container comprising or consisting of the viscoelastic component of any embodiment of the first aspect of the invention; and (b) a second container consisting of the ionically conductive aqueous solution of any embodiment of the composition of the first aspect of the invention.

This aspect of the invention can be used, for example, to prepare the compositions of the first aspect of the invention. In a preferred embodiment, the volume ratio of (a) to (b) in the containers is between about 1:1 and about 1:10.

In a fourth aspect, the present invention provides kits comprising (a) a first container comprising or consisting of the composition of any embodiment of the first aspect of the invention; and (b) a second container consisting of the ionically conductive aqueous solution of any embodiment of the first layer of the second aspect of the invention.

This aspect of the invention can be used, for example, to prepare the devices of the second aspect of the invention.

In one preferred embodiment of the third and fourth aspects of the invention, the first and second containers are separate compartments in a single container, separated by a partition that can be removed (in whole or in part), to permit mixing of the components prior to application in the methods of the invention. In a further preferred embodiment, the first and second containers are made of glass.

In another preferred embodiment, the first and second containers are syringes, either a single syringe with a removable partition (for removal just prior to mixing) or separate syringes. In a further preferred embodiment, the syringes comprise needles of at most 23 gauge with a hole on the side to facilitate application to the lens by coming into a conventional surgical port implanted in the sclera next to the iris. The side hole on the needle instead of at the tip enables the deposition of a hydrophilic droplet right on the center of the lens.

As noted, the kits may comprise any embodiment or combination of embodiments of the components as recited in the first and second aspects of the invention.

In a fifth aspect, the present invention provides methods for coating a hydrophobic surface, comprising coating the hydrophobic surface with a device according to the second aspect of the invention, wherein either the first layer of the device, when present, is in direct contact with the hydrophobic surface, or the second layer of the device is in direct contact with the hydrophobic surface. The hydrophobic surface can be of suitable type, including but not limited to silicone, hydrophobic acrylic, any form of silicon dioxide, quartz or silicon substrates used for medical device implants and surgical supplies, shunts, and tubing; and eyewear, such as sports visors, eye glasses, and goggles, having, for example high impact resistance coating such as a silicate over an underlying polycarbonate substrate.

The methods of the invention can be used in conjunction with any procedure in which coating the devices of the invention on a hydrophobic surface are useful, including but not limited to acting as an antifogging agent (such as in IOL surgical procedures, and acting as “lubricious” agent on medical implants, shunts and surgical supplies to (a) minimize tissue trauma, (b) maximize bio-compatibility (c) increase healing by enhancing better irrigation and flow in adjacent tissue.

The methods of the invention may utilize any embodiment or combination thereof of the compositions, devices, and kits of the invention.

Thus, in one preferred embodiment, the methods comprise methods for processing an intra-ocular lens (IOL), comprising (a) optionally contacting a surface of the IOL with a first layer of an ionically conductive aqueous solution according to any embodiment of the second aspect of the invention; and (b) contacting the first layer, when present, or the surface, with a second layer comprising any embodiment of the composition of the first aspect of the invention.

In another preferred embodiment, the methods comprise methods for processing a hydrophobic surface (including but not limited to tubing, medical implants, shunts, and surgical devices) for implantation into a subject, comprising (a) optionally contacting a surface of the hydrophobic surface with a first layer of an ionically conductive aqueous solution according to any embodiment of the second aspect of the invention; and (b) contacting the first layer, when present, or the hydrophobic surface, with a second layer comprising any embodiment of the composition of the first aspect of the invention.

The device can be applied at any suitable time; it can be applied prior to implantation, months in advance, on a lens, and rehydrated in saline. As necessary, the device can be applied post implantation on any hydrophobic lenses. In another preferred embodiment, the device is applied just before surgery/implantation.

When a first layer is applied to a surface, the second layer can be applied at any suitable time after application of the first layer; preferably within 1 second to 5 minutes; even more preferably within about 5 seconds to about 60 seconds; or about 5 seconds to about 30 seconds; or about 5 seconds to about 10 seconds. For IOL implantation, the method may be used when an artificial lens is piggy-backed with a remaining natural lens.

Condensation (or “fogging”) on silicone IOL's after implantation is most severe when the doctors have to go back into the eye with the silicone lens already installed, for example to repair a detached retina. This is extremely hard to handle, the surgeon has to wipe the surface every 15 seconds with saline, rendering the surgery extremely difficult and unsatisfactory. See, for example, http://www.crstoday.com/PDF%20Articles/0805/CRST0805_F9_Huang_Patel .html; Eaton A M, Jaffe G J, McCuen B W II, Mincey G J. Condensation on the posterior surface of silicone intraocular lenses during fluid-air exchange. Ophthalmology. 1995; 102:733-736; and Carlson A N, Stewart W C, Tso P C. Intraocular lens complications requiring removal or exchange. Surv Ophthalmol. 1998; 42:417-437.

Prior to the present invention, efforts to render silicone condensation-free consisted of continuously wiping the silicone surface covered by droplets, every 15 second during surgery, and/or applying thick, unstable gels, including on silicone implants for applications other than cataract surgery.

Presently, doctors who implant IOLs do not use these high performance multifocal silicone IOLs if they feel the patient ever has to have eye surgery again, for example in the case for retinal detachment. This severely curtails the use of silicone IOLs, limiting their market penetration to 20%, because it is difficult to guarantee never having to need another eye surgery or never getting a shock that detaches the retina. For example, diabetic patients, which constitute one sixth of the US population in 2009, are at high risk for multiple eye surgery, have high rates of cataracts, and cannot use these lenses.

The methods of the present invention significantly inhibit condensation on silicone IOLs and avoids opacification of the eye lens observed in at least 25% of alternative IOLs. While not being bound by a specific mechanism of action, the inventors believe that application of the device to a hydrophobic silicone surface, such as a silicone IOL, creates a biocompatible nanophase on the IOL surface that can maintain a wetting and bonding surface layer comprising a web of polymer strands from the composition of the invention, thereby impeding condensation of the opaque water film.

The device can be applied prior to surgery (such as IOL surgery or implantation of hydrophobic medical devices), or in subsequent surgeries if, for example, a chronic eye condition, such as a detached retina, occurs.

In one preferred embodiment, an emulsion is prepared, just prior to application, with a dispersive viscoelastic. The mixing is preferably conducted in glass vessels. The first layer can be optionally applied first to the hydrophobic surface, followed by layering of the composition in emulsion form. Each layer applied to the a surface is a continuous layer, as noted above. The device can prevent and eliminate the almost instantaneous condensation of moisture (“fogging”) from live tissues during, for example, vitreo-retineal eye surgery when a silicone intraocular lens is placed or already in place in the human eye. The maintenance of a pure hydrophilic character to the surface of the silicone implanted lens maintains the optical clarity critical for the surgeon to conduct the surgery and adjust the vision of the patient to its full potential, instead of being blinded by uncontrolled fogging.

In one preferred embodiment, the device is placed on the posterior side of the lens. The weight of the device is small in comparison to the electrostatic interaction with the hydrophobic surface to maintain a uniform molecular film and avoid delamination, deformation and/or detachment by gravity. The layer(s) can be applied by syringe dispensing or the Langmuir-Blodgett method onto the hydrophobic surfaces.

All of the embodiments each of the aspects of the invention can be used together and are suitable for combining in any combination except where the context clearly dictates otherwise.

### EXAMPLES

### Example 1

### Direct Observations: Understanding of the Unique Two-Step Water Interaction with Silicone Surfaces and Why it Enhances Condensation

The behavior of silicone IOLs is very different from acrylic lenses and single compound materials because silicone is a composite material comprising insulating silicon dioxide and organic chain polymers.

When in contact with an aqueous solution, silicon dioxide with saturated bonds exhibits a two-step response instead of a single one that can be described as initially strongly hydrophobic, followed by the slower response of the polymers which exhibit a opposite, hydrophilic behavior. In other words, water molecules, initially interact much more weakly with the highly insulating silicon dioxide backbone than with each other, and ball up into strongly spherical droplets with a very high surface tension. However, once the spherical droplets are formed, the polymeric components, which are less insulating and allow for some electronic reorganization, start to interact with the droplets and allow them to stick electrostatically to the surface.

These two different molecular responses, one in a very short timescale, the other in a longer one, allows for the formation of strongly spherical droplets, followed by sticky water droplets instead of a simple uniformly dry (hydrophobic) or uniformly wet surface (hydrophilic) seen in single component materials.

We tested and characterized this mechanism, to select and optimize factors that enhance or decrease this two-step condensation of a very opaque film of individual droplets on silicone.

### Example 2

### Simple Contact Angles Measurements to Quickly Characterize Surface Condensation During Tests and Compare Between Lenses Materials and Surfaces Modifications

The first tests measured how strong the initial hydrophobic behavior is and detect modifications. Contact angle measurements were made to compares silicone and acrylic wetting responses, in lenses as received and after chemical oxidation. Silicone consistently exhibited contact angles below 90 degrees (˜80 degrees) while acrylic, which does not have such a severe condensation problem, exhibited angles well above 90 degrees (˜120 degrees). Thus, when condensing drops come into contact on these IOLs, droplets on the silicone IOLs trap air between their boundaries, while the flatter droplets on acrylic IOLs contact each other at their base. Thus, the latter are more likely to form a continuous, uniform film through which visibility is maintained without refraction changes due to the connecting the droplets boundaries.

### Example 3

### Simple Contact Angles Measurements to Quickly Characterize Surface Condensation During Tests and Compare Between Lenses Materials and Surfaces Modifications

Surface topography over a 10 μm×10 μm area of silicone and acrylic IOLs was measured by AFM. Silicone showed a surface roughness around 10 nm over a length-scale of roughness about <100 nm; acrylic showed a surface roughness around 20 nm over a length-scale of 2 μm. The surface smoothness is significantly better on silicone lenses, which may explain in part their superior optical properties. In counting droplets on across 6 mm lenses, about 100 are found, thus yielding about 60 μm between nucleation sites for droplets. The spacing between average small scale features on the silicone surface is less than 100 nm for features less than 10 nm high, and about 10 μm for larger narrow features. Hence, the surface topography is not a factor in nucleating the condensing droplets. Being unusually smooth, silicone may help maintain the stability of the final droplets. The acrylic film has a much more significant surface roughness, which may increase wetting by increasing the surface area and disrupting the droplets boundaries.

### Example 4

### The Role of Surface Chemistry: Study in an Artificial Eye Chamber Prototype to Measure Preliminary Condensation Times and Delay Using Different Surface Treatments: (1) Surface Cleaning (2) Emulsion Approach

Time lapse photography was used to image a simple polymeric eyeball used for surgery practice to simulate condensation on a lens. While the time measured in a simple “open” eye” for saline condensation on the lens does not replicate the temperature conditions at the lens without an iris on top of the IOL, it does provides a preliminary, relative time scale to compare condensation times. According to surgeons, the exact aspect of the condensed droplets on silicone IOLs observed with this apparatus mimics the condensation observed during surgery. This benchmark ensured that the simulated condensed droplets matched known observations in the field.

### Example 5

### The Role of Surface Electronics: Electronic Bond Modification and Electronic Carrier Enrichment: Preliminary Experiments

By using the preliminary set-up of Example 4, we compare the times the IOLs remain clear from condensation for different surface treatment using FDA approved fluids for ophthalmological applications, (1) saline, and (2) DUOVisc®. We tested systematically a variety of emulsions made with both fluids.

By making an emulsion of DuoVisc® in water (we compared saline and DI water) and forming a film with it in our lab, we increased the time the lens stays clear of droplets until the concentration of DuoVisc® in the emulsion reaches a ratio of 25 to 1 in saline, after which increasing the amount of DuoVisc® in the saline actually decreases the time the IOLs remains condensation-free. A similar change of behavior with dilution in the emulsion of viscoelastic fluid in water is also observed with salt-free 18 MegOhms deionized water. However, in that case, a slow increase in duration is attained up to dilution of 5:1 of water into DuoVisc.

This behavior could be explained by DuoVisc® droplets distribution and average spacing (in the micron range) in the emulsion. Their size and spacing combination could exhibit an optimum for our purpose of shielding condensing droplets from the silicone IOL surface or interfere with droplet nucleation in the 50 μm droplet size they exhibit in our tests and surgery.

### Example 6

### Resistance to Condensation of Coated IOLs

The resistance to condensation in the silicone intra-ocular-lens was demonstrated by partially coating a lens with our a device according to the invention using FDA approved saline BSS+0 and the specific viscoelastic polymer Ocucoat® used in surgery (20 mg of HPMC/mL; 80,000 Da; 400+/−1500 centistokes; pH 7.2+/−0.4; 285+/−32 milliOsmoles). The first layer is pure balanced saline (BSS PLUS®), the second layer is an emulsion of in a ratio of 7:1 (seven to one) of saline to Ocucoat®.

The bi-layer was applied in situ using two separate syringes and the lens observed for condensation for 23 minutes. The layers are applied as a thin coating as droplet application bring about optical distortion. A surgical tool can be used to flatten and thin the layer and avoid excess application. Two patches were left uncoated on the 6 mm lens: (1) a 3 mm crescent shaped area on the left side; and (2) a 1 mm circular one to the right. This is simply done by avoiding touching the surface with the material. The experiment was repeated 10 times for testing reliability.

The results demonstrated that (a) condensation occurred on untreated IOLs; (b) the absence of condensation on the bilayer treated area of the IOLs for 23 minutes; and (c) the bilayer was stable: the uncoated area did not grow in size, but remains with the same area,demonstrating that the bilayer is neither diluted nor affected by adjacent water condensation. This latter property is important as condensation on silicone typically grows from the periphery and progressively invades the center of the lens. Other methods using application of saline or a variety of viscoelastic gels to prevent or remove condensation resulted in shrinking coatings that are diluted by adjacent condensation and allow droplets to invade rapidly the whole are in tens of seconds.

Condensation within the uncoated area progressed as a fine mist of droplets on the lens within the first 10 seconds which grew throughout the 23 minutes exposure. The initial droplet size ranges around 0.03-0.05 mm. The condensed water film on the uncoated area at 10 second is thus around 0.03-0.05 mm thick. After 23 minutes, the droplets grew by about an order of magnitude to about 0.3-0.5 mm. The condensed water film on the uncoated area is thus around 0.3-0.5 mm as well

This ripening mechanism allows one to estimate the thickness of the water film condensed on the uncoated area, and gives an independent measure of the time scale of the exposure and the amount of condensed water the bi-layer can successfully withstand. Thus, this sequence of measurements show that after increasing the exposure of the bilayer to condensing droplets by an amount of about an order of magnitude, the bilayer remains stable.

### Example 7

86 KDa HPMC was solvated in deionized water at weight percentages ranging from 10 wt % to 0.0001 wt % and a Bausch & Lomb 5inm silicone lens was dipped in the various solvated HPMC compositions and tested for anti-fogging behavior.

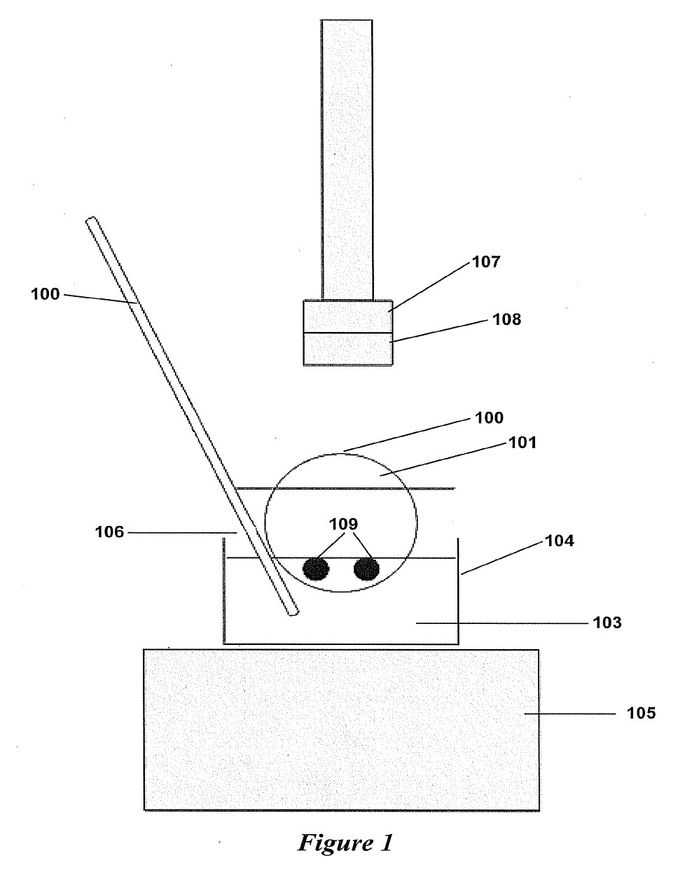

Coated lenses (100) were mounted on an artificial eyeball (101) in a small condensation chamber (102) as illustrated in FIG. 1. Water vapor is produced by heating water (103) in a petri dish (104) over a hot plate (105) to a temperature of 38° C., which was monitored by a mounted thermometer (106). The vapor reaches the surface of the lenses to simulate the condensation encountered in the human eye in surgery. Small holes (109) are present in the condensation chamber which is partially immersed in the 38° C. water heated in the petri dish. These holes are present in the condensation chamber supporting the lens to let water vapor from the heated water rise inside the condensation chamber and thus simulate condensation on the side of the lens facing down towards the inside of the eye in the same configuration and geometry encountered during surgery. The surface of the lenses was monitored by a camera (107) and optional microscope (108) mounted above the observed surface.

A minimum HPMC concentration for preventing lens fogging, for at least 20 minutes, was found to be 3×10⁻⁶ in w.t. which is equivalent to ½ monomers per nm². A marginal HPMC concentration was found to be about 1.3×10⁻⁵ to about 3×10⁻⁶ by w.t., or 0.013 wt %-0.0003 wt % which is equivalent to about ½ to 2 monomers per nm². An optical distortion cutoff HPMC concentration was found to be ≦1 wt %.

### Example 8

### Characterization of Coated Silica Wafers and the Silicone Lenses

Materials and Methods

De-ionized (DI) water was of 2 MΩ cm resistivity unless otherwise noted. Fused silica wafers were from Medtronic, and were cleaned via ultrasound agitation for 10 minutes in DI water of 18 MΩ cm resistivity. The silicone lenses used were Bausch & Lomb HD-500 intraocular lens (IOL). HPMC (C₃₂H₆₀O₁₉) CAS-9004-25-3, was 86 kDa molecular weight from Sigma-Aldrich and is hydrated using DI water.

Both the silica wafers and the silicone lenses were coated with HPMC film at room temperature, soaked in water-hydrated HPMC for 2 hours at various concentrations (from 0.20% w.t. to 1.00% w.t), then air dried under class 10K ventilation hood for a minimum of 24 hours.

Contact Angle and Roughness Measurements

Contact angle and surface tension were used to quantify water affinity utilizing Young's equation combined with Van Oss theory,

γ_{SL}=γ_{S}−γ_{L} cos θ 1)

γ=γ^{LW}+w\sqrt{γ⁺γ⁻} 2)

the modified Young-Dupré equation thus becomes

(1+cos θ)γ_{L}=2(\sqrt{γ_{S}^{LW}γ_{L}^{LW}}+\sqrt{γ_{S}⁺γ_{L}⁻}+{square root over (γ_{S}⁻γ_{L}⁺))} 3)

with γ_{L}=liquid's surface tension, γ_{S}=solid's surface tension, γ_{SL}=surface tension at solid-liquid interface, γ⁺ and γ⁻=Lewis acid-base component of surface tension, γ^{LW}=Lifshitz-van der Waals component of surface tension, θ=contact angle made at the liquid/solid interface. Large surface tension components or surface free energy result in the contact angle being small and surface being hydrophilic, while small surface tension results in the surface being hydrophobic. Although quartz silica and silicone surface free energy measurements have been conducted elsewhere, the fused quartz and medical silicone samples used in this paper need to be quantified and compared.

The Sessile drop method was used to conduct the contact angle measurement. The liquid droplets ranged in sizes from 0.4 μL˜5.0 μL and were delivered using a syringe with a 23 gauge cannula. The contact angle was measured using computer fitting techniques on the digital images. Both droplet size and contact angle were computed via computer processing. The surface topography is also known to affect the condensation/nucleation behavior of water.

Roughness and length-scale of roughness of surfaces were determined by tapping mode atomic force microscopy (TMAFM, Agilent). The AFM was operated in air with a silicon tip in AC mode. Gwyddion was the software used to produce images and roughness parameters: R_{q}=root mean square roughness, λₐ=average wavelength of the profile, and Δₐ=average absolute slope.

Rutherford Backscattering Spectroscopy

Areal density was used to analyze the HPMC film using He⁺⁺ Rutherford backscattering spectrometry (RBS). RBS has been used to analyze polymer and substrate profiles including diffusion profiles, the aging process of organic photovoltaic cells, and polymers used for medical purposes. Energy loss of ions can be used to determine the film thickness. Both the energy loss due to the in-path of incident α particles and the out-path of the scattered a particles need to be considered. Since Si exists in the substrate only and not in the film, one can measure the energy shift of the Si edge to determine the energy loss,

ΔE=kE₀−E 4)

with ΔE=energy loss, k=kinematic factor, E₀=incident ion energy, E=ion energy at the detector. Therefore to determine the areal density of the film,

$$Nt = \frac{{kE}_{0} - E}{k \frac{1}{\vert cos {θ}_{1} \vert} ( {ɛ}_{{E}_{0}} ) + \frac{1}{\vert cos {θ}_{2} \vert} ( {ɛ}_{{kE}_{0}} )} \qquad 5 )$$

where using Bragg's rule to compute the energy stopping factor,

$${ɛ}_{{C}_{32} {H}_{60} {O}_{19}} \approx \frac{32 {ɛ}_{C} + 60 {ɛ}_{H} + 19 {ɛ}_{O}}{32 + 60 + 19} \qquad 6 )$$

with θ₁=incident angle to sample normal and θ₂=detector angle to sample normal. ε=the stopping cross section of alpha particles in the HPMC. An incident angle of 0° (normal to the surface) was used for films prepared with of 0.33% -1.00% w.t. HPMC in water, while 65° incident angle was chosen for films prepared with <0.33% w.t. HPMC in water. The incident angles were chosen so that the silicon edge of the RBS spectra would be exposed for thicker films, while being able to enhance the energy loss resolution for thinner films. Detector angle is 170° and incident energy is 2 MeV with the beam current ˜20 nA.

HPMC consists of lighter C and O atoms which are typically difficult to detect on heavier substrates with a high degree of resolution. By implementing 4.265 MeV¹²C(α, α)¹²C and 3.045 MeV ¹⁶O(α, α)¹⁶O nuclear resonance scattering (NRS), one can determine the carbon versus oxygen composition at high resolution near the surface. Again in these measurements, the incident beam is normal to the sample; detector angle is 170°, and beam current is ˜20 nA.

The sample was separately measured for the height of the carbon and oxygen signals at 4.265 MeV and 3.045 MeV respectively. To maintain consistency, the Si signal was used to normalize all spectra,

$$\frac{{N}_{C} t}{{N}_{O} t} = \frac{\frac{{σ}_{Si} {\vert}_{E = C - Resonance}}{{σ}_{C} {\vert}_{E = C - Resonance}} \cdot \frac{{H}_{C}}{{H}_{Si}} {\vert}_{E = C - Resonance}}{\frac{{σ}_{Si} {\vert}_{E = O - Resonance}}{{σ}_{O} {\vert}_{E = O - Resonance}} \cdot \frac{{H}_{O}}{{H}_{Si}} {\vert}_{E = O - Resonance}} \qquad 7 )$$

with the subscripts of Si, O, and C representing silicon, oxygen, and carbon respectively,

$$\frac{{H}_{C}}{{H}_{Si}} and \frac{{H}_{O}}{{H}_{Si}} = normalized yield over Si signal ,$$

σ_{C} and σ_{O}=scattering cross section at resonance, N_{C}^{t} and N_{O}^{t}=areal density of carbon and oxygen in HPMC film. The carbon and oxygen signal heights, respectively, were extracted from the RBS spectra by performing a linear fit to the background silicon signal and then subtracting the fitted background.

Elastic recoil detection (ERD) of the hydrogen atom was conducted to provide a comparative method to the above areal density measurement, as well as determining the hydrogen composition ratio. Incident α particles at 2.8 MeV with a 10.6 μm mylar filter at the detector are used. Projectile angle is 75° in reference to the sample normal and elastic recoil detector angle is 30°. Once again the beam current was kept at ˜20 nA. RUMP simulation was then implemented to determine the areal density (Nt) of the HPMC film and the relative hydrogen composition compared to oxygen and carbon.

Ion beam damage in polymer films is a well known and impeding issue. Therefore it was necessary to construct damage curves, which can then be used to extrapolate the effective non-damaged yield in the subsequent areal density measurement using regression modeling.

Sample charging due to the ion beam incidence is observed for both silica wafer and silicone lens samples. Grounding with an aluminum foil wrapping with a 4 mm˜10 mm diameter hole minimizes the charging effect; enabling consistent, reproducible data collection. However, the charging effect still was significant enough to require an offset in terminal voltage to compensate for the required resonance energy; and hence this offset was introduced after adding the conductive wrapping. Resonance yield was not stable at the fixed carbon resonance of 4.265 MeV and oxygen resonance of 3.045 MeV. Therefore, the scattering yield was maximized around 4.265 MeV and 3.045 MeV by adjusting the offset due to the charging effect to obtain the resonance energy for collection of the spectra. The experiment was conducted in vacuum of 10⁻⁷ torr˜10⁻⁶ ton.

Condensation Behavior Versus Surface Free Energy and Surface Topography

The contact angle and related surface free energy components for silica and silicone samples are shown in Table 4. Surface free energy of silica is greater than that of silicone, particularly the Lewis basic component γ⁻. This results in silica's hydrophilic behavior versus silicone's hydrophobic behavior, which is shown by their respective contact angles.

Table 5 lists the roughness parameters of the respective samples. Notably, the roughness of the silicone is 9 times greater than that of the silica and the slope of features in silicone is about 7 times greater. However, the topographical wavelength of both samples is in the same range. Therefore, silicone provides more nano-scale capillary features

TABLE 4
Surface Energies of Silicone and Silica Correspond to the
Hydrophobic and Hydrophilic Behavior of the Respective Surfaces.

| | γ^{LW} | γ⁺ | γ⁻ | |
|---|---|---|---|---|
| Type | (mJ/m²) | (mJ/m²) | (mJ/m²) | Contact Angle with Water θ° |
| PDMS | 19.4^{a} | 0.8^{a} (γ^{polar}) | | 107.2^{a} |
| Silicone | | | | 104.7 ± 1.4 (from this work) |
| Silica | 41.3^{b} | 2.21^{b} | 35.68^{b} | 30.77 (calculated using |
| | | | | Equation (3)) |
| | | | | 32.2 ± 1.9 (from this work) |

^{a}Diversified Enterprises, Critical Surface Tension and Contact Angle with Water for Various Polymers (2009).
^{b}A. Zdziennicka, J. of Colloid and Interface Science, 340, 243-248 (2009).

TABLE 5
Roughness Parameters via AFM.

| Surface Type | R_{q} (nm) | λₐ (μm) | Δₐ |
|---|---|---|---|
| Silicone | 3.7 (2) | 0.28 (3) | 0.067 (4) |
| Quarts | 0.42 (5) | 0.22 (3) | 0.0094 (8) |
| HPMC 1% on Silicone | 0.31 (1) | 0.16 (1) | 0.0098 (4) |

From Tables 4 and 5 we can explain the differences in the condensation behavior that silicone and silica experience which further show how the morphology and water affinity affect condensation behavior. Condensation behavior was examined by placing coated and uncoated substrates above a water bath held at 70° C. Silicone is much rougher than silica, and hence one can imagine that each peak-valley-peak as a capillary-like structure. This allows for almost immediate condensation. However, because the roughness effectively isolates each condensed water drop, the total condensation area remains finely fogged from a macroscopic view. Since the surface free energy is relatively low compared to the silica, the condensed water drops tend to form water “beads”. And, nucleated drops tended to remain in place and coalescence is much slower than silica.

The condensation behavior on the silica was quite different. Because of its smoothness, it took much longer than the silicone for water droplets to nucleate on its surface. However, once these droplets were nucleated, due to the high surface free energy, the resulting condensation then tended to coalesce faster than that on the silicone. As a result, instead of a fine mist like fog as seen on the silicone, the condensation rapidly takes on the form of “puddles” of water spaced over the silica.

HPMC polymer coating was used to alter the condensation behavior of silica and silicone. TMAFM was used to determine how the HPMC coating modified the surface topography of the respective silica and silicone substrates. Roughness parameters in Table 5 demonstrate the wavelength was reduced significantly. HPMC is a water hydratable polymer which forms a water cage around its polymer chain via hydrogen bonding and has a high level of water affinity. The condensation behavior rapidly forms a complete wetting layer.

Polymer Film Composition and Areal Density Measurement

HPMC polymer film was characterized by areal density measurements via ion beam energy loss in conjunction with the respective damage curve extrapolation. Damage curves were obtained for Si0₂ (silica) and silicone substrates with polymer coatings from concentrations from 0.2% to 1.0%. The HPMC areal density was obtained by measuring the energy shift as defined in Equation (5) above, with various incident α particle dosages at 2 MeV to obtain the damage curve. Then the damage curve was fitted using exponential regression modeling. The concept and implementation of the damage curve was necessary since even small doses of α particles cause a reduction in polymer film areal density measurement. Using damage curves allows for the use of IBA as an analysis tool on films that are significantly damaged in the measurement process.

These results demonstrated that HPMC films with an areal density ranging from 10¹⁸ atom/cm²˜10¹⁹ atom/cm² can effectively prevent fogging on the silica and silicone surfaces by forming a complete wetting layer to improve visual clarity. A film that is too thick may cause visual distortion, while too thin of a film may have a wide range of effects, from merely experiencing initial heavy fogging and then clearing, to not being effective at all due to complete fogging, or a combination of these effects.

The HPMC polymer film carbon and oxygen ratios near the film's surface was determined by 4.265 MeV ¹²C(α, α)¹²C and 3.045 MeV ¹⁶O(α, α)¹⁶ O nuclear resonance scattering (NRS). Damage curves were used to compensate for the ion beam damage. Using the method specified in Equation (8) above, the resulting measured ratio gives,

$$\frac{{N}_{C}^{HPMC}}{{N}_{O}^{HPMC}} \approx 1.64 \pm 0.09 \qquad 8 )$$

which is in excellent agreement with the stoichiometric ratio of

$$\frac{{C}_{32}}{{O}_{19}} \approx 1.68 .$$

2.8 MeV elastic recoil detection (ERD) of hydrogen was fitted by RUMP simulation. The areal density is 11600 (10¹⁵ atom/cm²), with increments of 100 (10¹⁵ atom/cm²) for each simulation step. The areal density measured by ERD is in excellent agreement with the areal density as extracted by the Si signal energy loss method, which gave a range of 11500˜12500 (10¹⁵ atom/cm²) as an areal density. The hydrogen composition to oxygen and carbon ratio was also determined to be N_{C}:N_{O}N_{H}=1.64:1.00:2.70 with N_{H} step of 0.05, compared to the HPMC stoichiometric ratio of 1.68:1.00:3.16. The hydrogen content is 15% less than the bulk HPMC stoichiometric ratio would suggest. This discrepancy is suspected to be from the loss of hydrogen atom due to the ion beam damage.

Characterization of a solid surface's water affinity via contact angle measurement using the Sessile Drop method, coupled with surface analysis using TMAFM, help explain the behavior of water condensation during liquid nucleation and condensation. Polymer adsorption on solid surfaces alters the water affinity, as well as the topography of the surface. Characterization of the polymer modified surface via IBA and TMAFM is correlated to the resulting change in behavior of water condensation and formation of wetting layer on the HPMC polymer surface.

The development of the damage curve was essential in enabling the use of IBA as a tool to characterize HPMC polymer, and allows for the compensation of the damage which the HPMC experiences during the IBA process. NRS and ERD, in combination with standard RBS, enable for the high resolution areal density measurement and composition determination of the HPMC film. TMAFM, along with the determination of surface free energy via the Sessile Drop method open up a new methodology to characterize and explain surface interactions in terms of surface free energy and surface topography.

Characteristics of the surface and the polymer film have been successfully quantified in this work. However, the condensation pattern is still qualitative in nature. Future work will involve quantifying this condensation pattern and relating it to surface characteristics, thus creating a truly predictive model for water condensation.

### Example 9

Water condensation is a concern for many types of vision ware such as sports visors, eye glasses, goggles etc. To extend the present concepts to vision ware, one has to understand the complexity of the eyewear surfaces, commonly coated with high impact resistance coating such as silicate, and the underlying substrate, typically a form of polycarbonate. PIXE was used to help characterize and quantify the substrates (silica and factory treated polycarbonate) and the HPMC film. PIXE has been used by previous labs and organizations to analyze such diverse samples. Differential PIXE was used to determine the depth profiling and depth depletion assuming a homogeneous or layered profile structure. Differential PIXE was also explored as a method to identify the areal density of the film by using the substrate's (on which the film is applied) Si K X-ray emission. Note that the film has no Si, and hence the substrate and film are distinguishable.

Materials and Methods

De-ionized (DI) water was 2 MΩ cm resistivity unless otherwise noted. Fused silica wafers were from Medtronic, and were sonicated 10 minutes in DI water of 18 MΩ cm resistivity. Hydroxypropyl methylcellulose (HPMC), C₃₂H₆₀O₁₉ CAS-9004-25-3, was 86 kDa molecular weight from Sigma-Aldrich and was hydrated using DI water. Glycerin was from Sigma-Aldrich (G2289, CAS number 56-81-5), as was u-bromonaphthalene (17640, CAS number 90-11-9). The visors are Oakley brand and were selected randomly.

Both silica and the Oakley visors were treated with HPMC film at room temperature, soaked in water-hydrated HPMC for 2 hours at various concentrations (from 0.33% w.t. to 1.00% w.t.), and then air dried under a class 10K ventilation hood for a minimum of 24 hours.

Techniques

Contact angle measurements have been used to quantify water affinity and surface free energy utilizing Young's Equation combined with Van Oss theory. Large surface tension components result in the contact angle being small and surface being hydrophilic due to the strong intermolecular forces between the surface and water. Small surface tension components result in the surface being hydrophobic. The three surface tension components of solid surfaces from Van Oss theory can be calculated by measuring the contact angles using three distinct types of test liquids to the solid surface. One can then calculate surface tension through the following,

$$( \begin{matrix}\sqrt{{γ}_{S}^{LW}} \\ \sqrt{{γ}_{S}^{+}} \\ \sqrt{{γ}_{S}^{-}}\end{matrix} ) = \frac{1}{2} \cdot {( \begin{matrix}\sqrt{{γ}_{L 1}^{LW}} & \sqrt{{γ}_{L 1}^{-}} & \sqrt{{γ}_{L 1}^{+}} \\ \sqrt{{γ}_{L 2}^{LW}} & \sqrt{{γ}_{L 2}^{-}} & \sqrt{{γ}_{L 2}^{+}} \\ \sqrt{{γ}_{L 3}^{LW}} & \sqrt{{γ}_{L 3}^{-}} & \sqrt{{γ}_{L 3}^{+}}\end{matrix} )}^{- 1} \cdot ( \begin{matrix}( 1 + cos {θ}_{L 1} ) {γ}_{L 1} \\ ( 1 + cos {θ}_{L 2} ) {γ}_{L 2} \\ ( 1 + cos {θ}_{L 3} ) {γ}_{L 3}\end{matrix} ) \qquad 9 )$$

with θ=contact angle made at the liquid/solid interface, γ=surface tension, subscript of S=solid, L1-L3 represent the three types of liquid respectively, the superscript of γ⁺ and γ⁻=Lewis acid-base component of surface tension, and γ^{LW}=Lifshitz-van der Waals component of surface tension. This approach to analyzing surface free energy will be used to quantify the water affinity of the vision ware samples used in this paper.

The Sessile drop method was used to conduct the contact angle measurements. The liquid droplets' size ranged from 0.3 μL˜10 μL and was delivered using a syringe with a 23 gauge cannula. The contact angle was measured using computer fitting techniques for the digital images. Both droplet size and contact angle were computed via computer processing. The three components including Lewis acid-base and Lifshitz-van der Waals components of surface tension γ_{S}⁺, γ_{S}⁻, and γ_{S}^{LW} from Equation (10) above were calculated using three distinct types of test liquids: water, glycerin, and a-bromonaphthalene. The energy calculation tool was provided by SurfTen 4.3.

Roughness and length-scale of the roughness of surfaces were determined by tapping mode atomic force microscopy (TMAFM, Agilent). The AFM was operated in air with a silicon tip in AC mode. Gwyddion was the software used to produce images and roughness parameters; R_{q}=root mean square roughness, λₐ =average wavelength of the profile, and Δₐ=average absolute slope.

Rutherford backscattering spectrometry (RBS) combined with RUMP software provided composition and areal density measurement for the visor substrate as well as the polymer film areal density as a comparison to the PIXE method described below. 2 MeV H⁺⁺ with incident angle of 8° and detector angle of 170° together with RUMP simulation were used to identify the composition; incident He⁺⁺ at 2.8 MeV with incident angle of 8° and detector angle of 170° together with RUMP simulation was used to determine the areal density of the Si layer. HPMC stoichiometry near the surface was previously determined via 4.265 MeV ¹²C(α, α)¹²C, 3.045 MeV ¹⁶O(α, α)¹⁶O nuclear resonance scattering (NRS), and 2.8 MeV elastic recoil detection (ERD) of hydrogen.

Particle-Induced X-Ray Emission

Areal density measurements via incident α particle energy loss was used to analyze the HPMC film areal density on both the silica and visor substrates. Since silicon exists only in the substrate, the ratio of the number of silicon K X-rays detected and the amount of incident α particles is related to the areal density of silicon in the substrate accessible by the α particles through the following relation,

$${( ρ t )}_{z} = \frac{{N}_{x}}{{N}_{i}} {F}_{x , z} \qquad 10 )$$

with (ρt)_{Z} in g/cm²=areal density of element Z, N_{X}=number of X-rays, Nᵢ=number of incident ions, and F_{X, Z} is a constant for a given element, transition, primary ion beam, detector, and geometry. At certain incident energies, α particle can penetrate through a limited film depth into the Si-based substrate due to the energy loss via the in-path of the HPMC film and Si-based substrate. The amount of incident α particles is then compared to the amount of emitted silicon K X-rays with and without the HPMC film. Hence one can calculate the energy loss due solely to HPMC in-path where,

$$Δ {E}_{in - path} = {E}_{0} - E \approx [ \frac{1}{\vert cos {θ}_{in} \vert} {( ɛ )}_{{E}_{in - path}} ] Nt ( HPMC ) \qquad 11 )$$

where E₀ is the incident α particle energy just before striking the HPMC film, E is the energy of the α particle at the substrate interface Nt (HPMC) is equal to the areal density of the HPMC film, θᵢₙ the incoming angle of the α particles to the sample normal, and ε_{E}_{ᵢₙ₋ₚₐₜₕ} the energy loss cross section of HPMC. Incident energy used is between 1.4 MeV˜2.2 MeV. Since the number of incident in-path α particles backscattered by the HPMC layer at the given energy level is negligible (<10⁻⁵ Nᵢ), the silicon K X-ray attenuation due to out-path of HPMC layer attenuation is between 0.5%˜5.0% for 10¹⁸ atom/cm²˜10¹⁹ atom/cm² of the HPMC film if the detector is at the sample normal. Thus, one should expect with the systematic error, yields of the actual areal density are lower than this measurement. The experiment is conducted in a vacuum of 10⁻⁷ torr˜10⁻⁶ torr without a filter. The incident angle is 8° and detector angle is 37° to the sample normal for silica samples; and an incident angle of 30° and detector angle of 75° to sample normal for visor samples since the large incident angle is necessary due to the thinner silicate layer on visor samples. Controlling the beam current is critical to minimize detector dead time as well as pileup. Detection of X-rays was most consistent when the beam current <0.2 nA, therefore data collection was taken at 0.1 nA. Each spectrum received a charge of 0.5 μC. Sample charging due to the incident particles was also noticed for both the silica wafer and visor samples. Using an aluminum foil wrapping with a 4 mm˜10 mm diameter hole minimized the charging effect and stabilized the spectra; enabling consistent, repetitive data collection. GUPIX was used to calculate the area count of the silicon K X-rays.

Composition analysis of the Oakley visors utilized proton incident particles. Incident energy is at 1.8 MeV, incident angle is 40° and detector angle is 40° to sample normal in a vacuum of ˜10⁻¹ torr without filter, and a beam current <0.3 nA.

### Polymer Film Areal Density on Silica

The relationship of incident α particle energy E was plotted against the ratio of detected silicon K X-ray count over the incident particle count

$$\frac{{N}_{x}}{{N}_{i}}$$

for silica and Oakley visor substrates without HPMC film by varying the incident energy was used as the baseline for the relative yield graph. The log-log regression modeling uncertainty was analyzed to serve as the basis to obtain the error of the areal density calculation utilizing the differential PIXE method. Areal density via the RBS method was obtained from RUMP simulation of the spectra. The uncertainty was analyzed from the roughness of determined by analyzing the Si leading edge of which indicated the roughness of the film was observed. Areal density of the HPMC film and comparison to its RBS measurement are shown in Table 6. The measurements of the areal density by PIXE and RBS are in agreement.

TABLE 6
Area density results of HPMC polymer film on silica substrate.

| | Nt (10¹⁵ atom/cm²) | Nt (10¹⁵ atom/cm²) |
|---|---|---|
| HPMC gel concentration | He⁺⁺ PIXE | He⁺⁺ RBS |
| 1.00% w.t. | 7500 ± 500 | — |
| 0.50% w.t. | 4270 ± 560 | 5000 ± 1000 |
| 0.40% w.t. | 3040 ± 580 | 2700 ± 700 |
| 0.33% w.t. | 2100 ± 600 | — |

Once the substrate relative yield graph was set, areal density measurement was straight forward using this method. Another advantage of this method, when compared to using RBS, is that thicker films can be handled without the elevation of the incident particle energy. However, the thicker the film, the X-ray attenuation due to the out-path of HPMC becomes more significant. Results from both methods are in-line with similar order of resolution.

### Visor High Impact Resistance Coating

The proton PIXE spectra demonstrated that there is significant Si and S on both the convex and concave sides of the visor. RUMP simulation of the RBS spectra confirmed the presence of the silicon and sulfur in the estimated stoichiometric formula of SiO₂C_{2.5} with an areal density of 1.3×10¹⁹ atom/cm². Therefore, in order to utilize the energy loss method for areal density measurement, the incident α particle angle was increased to 75° to make the in-path longer, to ensure a large enough silicon thickness.

Analysis of the surface topography on the convex and concave sides of the visors was done using TMAFM. Both concave and convex sides have similar root mean square roughness of ˜1 nm (Table 7), which provides nucleation sites for water condensation. Contact angle and surface free energy information is shown in Table 8 and 9, and leans slightly hydrophilic. The roughness parameter of a typical feature was ˜1 nm and average wavelength of 0.2 μm˜0.3 μm, provides significant nucleation sites for rapid condensation on the area without the HPMC film. A slightly hydrophilic surface will indicate that the surface condensation pattern will be puddle-like upon ripening. Thus the combination of surface topography and surface free energy predicts the type of water condensation.

TABLE 7
Summary of the convex and concave roughness parameters.

| Roughness Parameters | R_{q} (nm) | λₐ (μm) | Δₐ |
|---|---|---|---|
| Concave | 1.14 (9) | 0.27 (1) | 0.0201 (8) |
| Convex | 1.49 (15) | 0.22 (2) | 0.032 (2) |
| Concave with 0.5% HPMC film | 0.54 (2) | 0.33 (5) | 0.009 (1) |

TABLE 8
Contact angle measurement for the Oakley visor.

| Surface | θ_{water}° | θ_{glycerin}° | θ_{α-bromonaphthalene}° |
|---|---|---|---|
| Concave side | 61.5 ± 1.4 | 54.7 ± 0.4 | 32.2 ± 1.6 |
| Convex side | 74.5 ± 1.0 | 78.4 ± 1.7 | 36.7 ± 1.5 |

TABLE 9
Surface free energy measurement for the Oakley visor.

| | Surface tension | | | |
|---|---|---|---|---|
| Surface | (mJ/m²) | γ^{LW} (mJ/m²) | γ⁻ (mJ/m²) | γ⁺ (mJ/m²) |
| Concave side | 40.6~49.7 | 37.8 ± 1.7 | 16.8 ± 5.3 | 0.7 ± 0.4 |
| Convex side | 34.2~47.8 | 36.0 ± 1.8 | 18.0 ± 4.8 | 0.4 ± 0.5 |

### Polymer Film Areal Density on Visor

The HPMC polymer film on the visor altered the surface topography, as well as water affinity and thus altered the condensation behavior. Roughness parameters are noted in Table 7. The hydratable polymer mesh provides high water affinity which enhances the coalescence of the nucleated droplets. Roughness scaling from several angstroms to several nm will provide ample nucleation sites, which will induce rapid condensation. The combined topography of the aforementioned characteristics leads to a rapid condensation which is immediately followed by coalescence, and the subsequent elimination of the fogging.

HPMC film areal density measurement and comparison to RBS using the energy loss method described above is shown in Table 10. The results show that the HPMC film with areal density ranging from 10¹⁸ atom/cm²˜10¹⁹ atom/cm² can effectively prevent fogging on the visor surfaces by forming a complete wetting layer to improve visual clarity. However, initial fogging was noticeable while the wetting layer was formed. It was also observed that the HPMC film on the visor surface had a larger wavelength than the HPMC film on the silica wafer. After the initial fogging, rehydration of the polymer formed a complete wetting layer and no further fogging was observed. Finally, from Table 10, the areal density derived from PIXE is of the same order as that derived from RBS; however, the result differences of the visor substrate were more significant than that of the silica substrate. We noticed that the stoichiometric variations in the RBS samples ranging from SiO₂C_{2.5} to SiO₃C₃, indicating slight compositional fluctuations at different sample locations of the visor coating may be the cause of error from the PIXE method, while the RBS result uncertainty indicates roughness of the HPMC. Hence, the respective areal density differences may be due to the observed variations.

TABLE 10
Area density results of HPMC polymer film on visor substrates.

| | Nt (10¹⁵ atom/cm²) | Nt (10¹⁵ atom/cm²) |
|---|---|---|
| HPMC gel concentration | He⁺⁺ PIXE | He⁺⁺ RBS |
| 1.00% w.t. | 16560 ± 170 | — |
| 0.50% w.t. | 9430 ± 150 | 6500 ± 500 |
| 0.33% w.t. | 1430 ± 170 | 2400 ± 400 |
| 0.20% w.t. | 1640 ± 160 | 2000 ± 300 |

Areal density measurements using differential He⁺⁺ PIXE were used to successfully characterize the HPMC film on Si based surfaces. The substrate allowed for the generation of a reference graph based on incident particle energy, which was then applied to the areal density measurement. The a-PIXE was necessary due to the thickness and composition of both the HPMC film and the Si based substrate involved.

The high impact resistance silicate coating, with Si and S, were characterized using proton PIXE, He⁺⁺ RBS, TMAFM, and contact angle measurement. The results established the relation between the coating's condensation behavior, its surface free energy, topography, and hydrophilic/hydrophobic behaviors.

Polymer adsorption on the visor coating altered the surface water affinity, as well as the topography of the surface, and hence alters the water condensation behavior. The addition of an HPMC film on the Oakley visors helps to minimize the fogging problem.

Control of fogging on sports visors is effective via HPMC polymer adsorption. However, it was observed that the HPMC film wavelength was larger for sports visors than the film wavelength for the silica wafer.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby. Each referenced document herein is incorporated by reference in its entirety for all purposes.

Changes can be made in the composition, operation and arrangement of the method of the present invention described herein without departing from the concept and scope of the invention as defined in the following claims.

We claim:

1. A composition, comprising a mixture of:
(a) a solvated viscoelastic polymeric gel, comprising a viscoelastic polymer having a molecular weight of between about 20,000 Da and about 4,000,000 Da; and
(b) an ionically conductive aqueous solution;
wherein the volume ratio of (a) to (b) is between 1:1 and 1:10.

2. The composition of claim 1, wherein the composition is an emulsion.

3. The composition of claim 1 wherein the ionically conductive aqueous solution comprises at least 0.03% electrolytes.

4. The composition of claim 1 wherein the ionically conductive aqueous solution approximates the composition of human vitreous humor.

5. The composition of claim 1 wherein the solvated viscoelastic polymeric gel comprises a viscoelastic polymer having a molecular weight of between about 20,000 Da and about 500,000 Da.

6. The composition of claim 1 wherein the solvated viscoelastic polymeric gel, comprising a viscoelastic polymer having a molecular weight of between about 20,000 Da and about 200,000 Da.

7. The composition of claim 1 wherein the solvated viscoelastic polymeric gel is homogenous.

8. The composition of claim 1, wherein the solvated viscoelastic polymeric gel comprises hydroxypropylmethylcellulose.

9. The composition of claim 1 comprising between 0.0003 wt % and 10 wt % of the viscoelastic polymer.

10. A device, consisting of
(a) an optional first layer comprising an ionically conductive aqueous solution; and
(b) a second layer comprising the composition of claim 1;
wherein when the first layer is present, the first layer and the second layer are in direct contact.

11. The device of claim 10 disposed on a hydrophobic surface of a hydrophobic substrate, wherein the second layer is in direct contact with the hydrophobic surface is a continuous layer.

12. The device of claim 10 disposed on a hydrophobic surface of a hydrophobic substrate, wherein the first layer is in direct contact with the hydrophobic surface, and wherein each of the first layer and the second layer are continuous layers.

13. The device of claim 11, wherein the hydrophobic substrate is selected from the group consisting of silicone, hydrophobic acrylic, polycarbonate, silicon dioxide, quartz, and silicon substrates used for medical device implants, surgical supplies, shunts, tubing, intraocular lens; and eyewear.

14. The device of claim 11, wherein the hydrophobic substrate is an intraocular lens.

15. The device of claim 10, wherein the volume of each layer is at least 0.5 μL.

16. The device of claim 10, wherein the device has a density between about 0.5 monomers/nm² and about 2000 monomers/nm²

17. The device of claim 10, wherein the device has an areal density of between about 10¹⁸ atom/cm² and about 10¹⁹atom/cm² as measured by He⁺⁺ Rutherford backscattering spectrometry (RBS).

18. A kit comprising
(a) a first container comprising a solvated viscoelastic polymeric gel, comprising a viscoelastic polymer having a molecular weight of between about 20,000 Da and about 4,000,000 Da; and
(b) a second container consisting of an ionically conductive aqueous solution.

19. A kit comprising
(a) a first container comprising (i) a solvated viscoelastic polymeric gel, comprising a viscoelastic polymer having a molecular weight of between about 20,000 Da and about 4,000,000 Da; and (ii) an ionically conductive aqueous solution; wherein the volume ratio of (a) to (b) is between 1:1 and 1:10.
(b) a second container consisting of an ionically conductive aqueous solution.

20. The kit of claim 18, wherein the first and second containers are separate compartments in a single container, separated by a removable partition.

21. The kit of claim 18, wherein the first and second containers are syringes.

22. A method for coating a hydrophobic surface, comprising coating the hydrophobic surface with a device according to claim 10, wherein either the first layer of the device, when present, is in direct contact with the hydrophobic surface, or the second layer of the device is in direct contact with the hydrophobic surface.

23. A method for processing an intra-ocular lens (IOL), comprising
(a) optionally forming a first layer on a surface of the IOL by contacting the surface with an ionically conductive aqueous solution; and
(b) contacting the first layer, when present, or the surface, with a second layer comprising (i) a solvated viscoelastic polymeric gel, comprising a viscoelastic polymer having a molecular weight of between about 20,000 Da and about 4,000,000 Da; and (ii) an ionically conductive aqueous solution; wherein the volume ratio of (a) to (b) is between 1:1 and 1:10.

24. A method for processing a hydrophobic surface for implantation into a subject, comprising
(a) optionally forming a first layer on a surface of a hydrophobic surface by contacting the surface with an ionically conductive aqueous solution; and
(b) contacting the first layer, when present, or the hydrophobic surface, with a second layer comprising (i) a solvated viscoelastic polymeric gel, comprising a viscoelastic polymer having a molecular weight of between about 20,000 Da and about 4,000,000 Da; and (ii) an ionically conductive aqueous solution; wherein the volume ratio of (a) to (b) is between 1:1 and 1:10.
